(19) United States
(12) Patent Application Publication
(10) Pub. No.: US 2006/0276771 A1
(43) Pub. Date: Dec. 7, 2006

(54) System and method providing for user intervention in a diabetes control arrangement

(76) Inventors: Paul J. Galley, Cumberland, IN (US); Ajay Thukral, Indianapolis, IN (US); Siva Chittajallu, Indianapolis, IN (US); Robin Wagner, Fishers, IN (US); Stefan Weinert, Pendleton, IN (US); Steven A. Bousamra, Carmel, IN (US)

Correspondence Address: BARNES & THORNBURG LLP, 11 SOUTH MERIDAN STREET, INDIANAPOLIS, IN, 46204, US

(21) Appl. No.: 11/145,910

(22) Filed: Jun. 6, 2005

(51) Int. Cl. A61M 31/00

(52) U.S. Cl. 604/503; 604/66

(57) ABSTRACT

A system providing for user intervention in a medical control arrangement may comprise a first user intervention mechanism responsive to user selection thereof to produce a first user intervention signal, a second user intervention mechanism responsive to user selection thereof to produce a second user intervention signal, and a processor executing a drug delivery algorithm forming part of the medical control arrangement. The processor may be responsive to the first user intervention signal to include an intervention therapy value in the execution of the drug delivery algorithm, and responsive to the second user intervention signal to exclude the intervention therapy value from the execution of the drug delivery algorithm. The medical control arrangement may be a diabetes control arrangement, the drug delivery algorithm may be an insulin delivery algorithm, and the intervention therapy value may be, for example, an intervention insulin quantity or an intervention carbohydrate quantity.

### FIELD OF THE INVENTION

The present invention relates generally to diabetes control arrangements, and more specifically to systems and methods providing for user intervention in such diabetes control arrangements.

### BACKGROUND

Conventional diabetes control arrangements may be or include fully or semi closed-loop systems operable to determine and deliver insulin to users. It is desirable to allow user intervention in such systems to provide fail-safe operation.

### SUMMARY

The present invention may comprise one or more of the features recited in the attached claims, and/or one or more of the following features and combinations thereof. A system providing for user intervention in a diabetes control arrangement may comprise means responsive to user selection thereof for producing one of a first and a second user intervention signal, and a processor executing an insulin delivery algorithm forming part of the diabetes control arrangement. The processor may be responsive to the first user intervention signal to include one of an intervention insulin quantity and an intervention carbohydrate quantity in the execution of the insulin delivery algorithm. The processor may be responsive to the second user intervention signal to exclude the one of the intervention insulin quantity and the intervention carbohydrate quantity from the execution of the insulin delivery algorithm.

The processor may be configured to continue uninterrupted execution of the insulin delivery algorithm regardless of whether the first or second user intervention signal is produced.

The system may further include means for providing the one of the intervention insulin quantity and the intervention carbohydrate quantity to the processor.

The processor may be responsive to the first user intervention signal to process the intervention insulin quantity by adding the intervention insulin quantity to a current insulin bolus amount. The processor may further be responsive to the first user intervention signal to command administration of the combination of the intervention insulin quantity and the current insulin bolus amount to the user. The current insulin bolus amount may be a positive-valued insulin bolus amount. Alternatively, the current insulin bolus amount may be a zero-valued insulin bolus amount.

The processor may be responsive to the first user intervention signal to process the intervention carbohydrate quantity by modifying a blood glucose target as a function of the intervention carbohydrate quantity.

The system may further include a database having insulin delivery and intervention carbohydrate information stored therein. The processor may be responsive to either of the first and second user intervention signals to enter the one the intervention insulin quantity and the intervention carbohydrate quantity into the database.

The processor may be operable to wait for a delay time prior to including the one of the intervention insulin quantity and the intervention carbohydrate quantity in the execution of the insulin delivery algorithm.

A method of allowing user intervention in a diabetes control arrangement may comprise executing an insulin delivery algorithm forming part of the diabetes control arrangement, monitoring first and second user intervention mechanisms, including one of an intervention insulin quantity and an intervention carbohydrate quantity in the execution of the insulin delivery algorithm in response to user selection of the first user intervention mechanism, and excluding the one of the intervention insulin quantity and the intervention carbohydrate quantity from the execution of the insulin delivery algorithm in response to user selection of the second user intervention mechanism.

The method may further include receiving the one of the intervention insulin quantity and the intervention carbohydrate quantity.

The method may further include entering the one of the intervention insulin quantity and the intervention carbohydrate quantity into a database in response to user selection of either of the first and second user intervention mechanisms. The method may further include date and time stamping the one of the intervention insulin quantity and the intervention carbohydrate quantity prior to entry into the database.

The method may further include waiting for a delay time after the user selection of the first user intervention mechanism and prior to including the one of the intervention insulin quantity and the intervention carbohydrate quantity in the execution of the insulin delivery algorithm.

A system providing for user intervention in a medical control arrangement may comprise a first user intervention mechanism responsive to user selection thereof to produce a first user intervention signal, a second user intervention mechanism responsive to user selection thereof to produce a second user intervention signal, and a processor executing a drug delivery algorithm forming part of the medical control arrangement. The processor may be responsive to the first user intervention signal to include an intervention drug quantity in the execution of the drug delivery algorithm. The processor may be responsive to the second user intervention signal to exclude the intervention drug quantity from the execution of the drug delivery algorithm.

The system may further include means for receiving the intervention drug quantity.

The medical control arrangement may be a diabetes control arrangement, the drug delivery algorithm may be an insulin delivery algorithm, and the intervention drug quantity may be an intervention insulin quantity. The processor may be responsive to the first user intervention signal to include the intervention insulin quantity in the execution of the insulin delivery algorithm by adding the intervention insulin quantity to a current insulin bolus amount. The processor may further be responsive to the first user intervention signal to command administration of the combination of the intervention insulin quantity and the current insulin bolus amount to the user.

The system may further include a database having drug delivery information stored therein. The processor may be responsive to either of the first and second user intervention signals to enter the intervention drug quantity into the database. The processor may be configured to date and time stamp the intervention drug quantity prior to entry into the database.

The processor may be operable to wait for a delay time prior to including the intervention drug quantity in the execution of the insulin delivery algorithm.

The processor may be configured to continue uninterrupted execution of the insulin delivery algorithm regardless of whether the first or second user intervention signal is produced.

A method of allowing user intervention in a medical control arrangement may comprise executing a drug delivery algorithm forming part of the medical control arrangement, monitoring first and second user intervention mechanisms, including an intervention drug quantity in the execution of the drug delivery algorithm in response to user selection of the first user intervention mechanism, and excluding the intervention drug quantity from the execution of the drug delivery algorithm in response to user selection of the second user intervention mechanism.

The method may further include receiving the intervention drug quantity.

The method may further include entering the intervention drug quantity into a database in response to user selection of either of the first and second user intervention mechanisms. The method may further include date and time stamping the intervention drug quantity prior to entry into the database.

The method may further include waiting for a delay time after the user selection of the first user intervention mechanism and prior to including the intervention drug quantity in the execution of the drug delivery algorithm.

The medical control arrangement may be a diabetes control arrangement, the drug delivery algorithm may be an insulin delivery algorithm and the intervention drug quantity may be an insulin intervention quantity.

A system providing for user intervention in a medical control arrangement may comprise a first user intervention mechanism responsive to user selection thereof to produce a first user intervention signal, a second user intervention mechanism responsive to user selection thereof to produce a second user intervention signal, and a processor executing a drug delivery algorithm forming part of the medical control arrangement. The processor may be responsive to the first user intervention signal to include an intervention therapy value in the execution of the drug delivery algorithm. The processor may be responsive to the second user intervention signal to exclude the intervention therapy value from the execution of the drug delivery algorithm.

The system may further include means for receiving the intervention therapy value.

The medical control arrangement may be a diabetes control arrangement, the drug delivery algorithm may be an insulin delivery algorithm, and the intervention therapy value may be an intervention insulin quantity. Alternatively, the intervention therapy value may be an intervention carbohydrate quantity corresponding to a quantity carbohydrates recently intervention by the user. In the former case, the processor may be responsive to the first user intervention signal to include the intervention insulin quantity in the execution of the insulin delivery algorithm by adding the intervention insulin quantity to a current insulin bolus quantity. The current insulin bolus quantity may have a value greater than or equal to zero. In the latter case, the processor may be responsive to the first user intervention signal to include the intervention carbohydrate quantity in the execution of the insulin delivery algorithm by modifying a blood glucose target as a function of the intervention carbohydrate quantity.

The system may further include a database having therapy value information stored therein. The processor may be responsive to either of the first and second user intervention signals to enter the intervention therapy value into the database. The processor may be configured to date and time stamp the intervention therapy value prior to entry into the database.

The processor may be operable to wait for a delay time prior to including the intervention therapy value in the execution of the drug delivery algorithm.

The processor may be configured to continue uninterrupted execution of the drug delivery algorithm regardless of whether the first or second user intervention signal is produced.

A method of allowing user intervention in a medical control arrangement may comprise executing a drug delivery algorithm forming part of the medical control arrangement, monitoring first and second user intervention mechanisms, including an intervention therapy value in the execution of the drug delivery algorithm in response to user selection of the first user intervention mechanism, and excluding the intervention therapy value from the execution of the drug delivery algorithm in response to user selection of the second user intervention mechanism.

The method may further include receiving the intervention therapy value.

The method may further include entering the intervention therapy value into a database in response to user selection of either of the first and second user intervention mechanisms. The method may further include date and time stamping the intervention therapy value prior to entry into the database.

The method may further include waiting for a delay time after the user selection of the first user intervention mechanism and prior to including the intervention therapy value in the execution of the drug delivery algorithm.

The medical control arrangement may be a diabetes control arrangement, the drug delivery algorithm may be an insulin delivery algorithm and the intervention therapy value may be an insulin intervention quantity. Alternatively, the intervention therapy value may be an intervention carbohydrate quantity corresponding to a quantity carbohydrates recently intervention by the user.

### BRIEF DESCRIPTION OF THE DRAWINGS

FIG. 1 is a block diagram of one illustrative embodiment of a system providing for user intervention in a controlled insulin delivery arrangement.

FIG. 2 is a flowchart of one illustrative embodiment of a software algorithm for providing for user intervention in a controlled insulin delivery system.

FIG. 3 is a flowchart of one illustrative embodiment of the intervention insulin quantity processing routine called by the algorithm of FIG. 2.

FIG. 4 is a flowchart of one illustrative embodiment of the intervention carbohydrate quantity processing routine called by the algorithm of FIG. 2.

### DESCRIPTION OF THE ILLUSTRATIVE EMBODIMENTS

For the purposes of promoting an understanding of the principles of the invention, reference will now be made to a number of illustrative embodiments shown in the attached drawings and specific language will be used to describe the same.

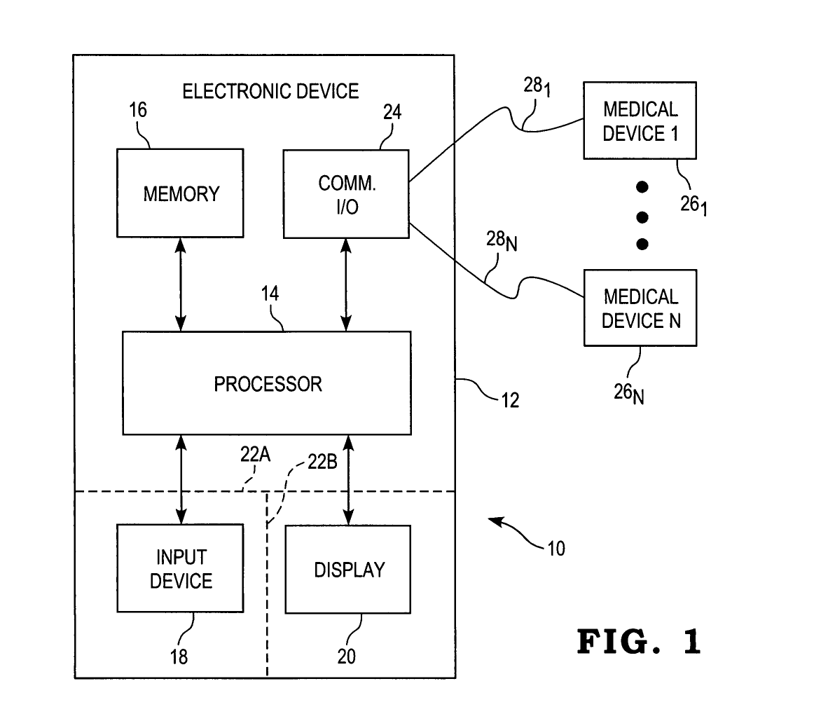

Referring now to FIG. 1, a block diagram of one illustrative embodiment of a system 10 providing for user intervention in a diabetes control arrangement is shown. In the illustrated embodiment, the system 10 includes an electronic device 12 having a processor 14 in data communication with a memory unit 16, an input device 18, a display 20 and a communication input/output unit 24. The electronic device 12 may be provided in the form of a general purpose computer, central server, personal computer (PC), lap top or notebook computer, personal data assistant (PDA) or other hand-held device, external infusion pump, or the like. The electronic device 12 may be configured to operate in accordance with one or more conventional operating systems including for example, but not limited to, windows, linux and palm OS, and may be configured to process data according to one or more conventional internet protocols for example, but not limited to, NetBios, TCP/IP and AppleTalk. In any case, the electronic device 12 forms part of a closed-loop or semi-closed loop diabetes control system, examples of which will be described hereinafter. The processor 14 is, in the illustrated embodiment, microprocessor-based, although the processor 14 may alternatively formed of one or more general purpose and/or application specific circuits and operable as described hereinafter. The memory unit 16 includes, in the illustrated embodiment, sufficient capacity to store data, one or more software algorithms executable by the processor 14 and other data. The memory unit 16 may include one or more conventional memory or other data storage devices.

The input device 18 may be used in a conventional manner to input and/or modify data. In the illustrated embodiment, the display 20 is also included for viewing information relating to operation of the device 12 and/or system 10. Such a display may be a conventional display device including for example, but not limited to, a light emitting diode (LED) display, a liquid crystal display, a cathode ray tube (CRT) display, or the like. Alternatively or additionally, the display 20 may be or include an audible display configured to communicate information to a user or third party via one or more coded patterns, vibrations, synthesized voice responses, or the like. Alternatively or additionally, the display 20 may be or include one or more tactile indicators configured to display tactile information that may be discerned by the user or a third party.

In one embodiment, the input device 18 may be or include a conventional keyboard or key pad for entering alphanumeric data into the processor 14. Such a keyboard or key pad may include one or more keys or buttons configured with one or more tactile indicators to allow users with poor eyesight to find and select an appropriate one or more of the keys, and/or to allow users to find and select an appropriate one or more of the keys in poor lighting conditions. Alternatively or additionally, the input device 18 may be or include a conventional mouse or other conventional point and click device for selecting information presented on the display 20. Alternatively or additionally, the input device 18 may include the display 20 configured as a graphical user interface (GUI). In this embodiment, the display 20 may include one or more selectable inputs that a user may select by touching an appropriate portion of the display 20 using an appropriate implement. Alternatively or additionally, the input device 18 may include a number of switches or buttons that may be activated by a user to select corresponding operational features of the device 12 and/or system 10. Alternatively or additionally, the input device 18 may be or include voice activated circuitry responsive to voice commands to provide corresponding input data to the processor 14. In any case, the input device 18 and/or display 20 may be included with or separate from the electronic device 12 as indicated by the dashed lines 22A and 22B.

In some embodiments, the system 10 may include a number, N, of medical devices 26₁-26_{N}, wherein N may be any positive integer. In such embodiments, any of the one or more medical devices 26₁-26_{N} may be implanted within the user's body, coupled externally to the user's body (e.g., such as an infusion pump), or separate from the user's body. Alternatively or additionally, one or more of the medical devices 26₁-26_{N} may be mounted to and/or form part of the electronic device 12. In the illustrated embodiment, the number of medical devices 26₁-26_{N} are each configured to communicate wirelessly with the communication I/O unit 24 of the electronic device via one of a corresponding number of wireless communication links 28₁-28_{N}. The wireless communications may be one-way or two-way. The form of wireless communication used may include, but should not be limited to, radio frequency (RF) communication, infrared (IR) communication, RFID (inductive coupling) communication, acoustic communication, capacitive signaling (through a conductive body), galvanic signaling (through a conductive body), or the like. In any such case, the electronic device 12 and each of the number of medical devices 26₁-26_{N} include conventional circuitry for conducting such wireless communications circuit 18₁ may further include, as appropriate. Alternatively or additionally, one or more of the medical devices 26₁-26_{N} may be configured to communicate with the electronic device 12 via one or more conventional hardwire connections therebetween. Each of the one or more medical devices 26₁-26_{N} may include any one or more of a conventional processing unit, conventional input/output circuitry and/or devices and one or more suitable data and/or program storage devices.

The system 10 illustrated in FIG. 1 is, or forms part of, a conventional closed-loop or semi closed-loop diabetes control arrangement. In this regard, the system 10 includes a delivery mechanism for delivering controlled amounts of a drug; e.g., insulin, glucagon, incretin, or the like, and/or offering an alternatively actionable recommendation to the user via the display 20, e.g., ingesting carbohydrates, exercising, etc. The system 10 may be provided in any of a variety of conventional configurations, and examples of some such configurations will now be described. It will be understood, however, that the following examples are provided merely for illustrative purposes, and should not be considered limiting in any way. Those skilled in the art may recognize other possible implementations of a closed-loop or semi-closed loop diabetes control arrangement, and any such other implementations are contemplated by this disclosure.

In a first example implementation of the system 10, the electronic device 12 is provided in the form of a conventional insulin pump configured to be worn externally to the user's body and also configured to controllably deliver insulin to the user's body. In this example, the number of medical devices 26₁-26_{N} may include one or more implanted sensors and/or sensor techniques for providing information relating to the physiological condition of the user. Examples of such implanted sensors may include, but should not be limited to, a glucose sensor, a body temperature sensor, a blood pressure sensor, a heart rate sensor, or the like. In implementations that include an implanted glucose sensor, the system 10 may be a fully closed-loop system operable in a conventional manner to automatically monitor blood glucose and deliver insulin, as appropriate, to maintain blood glucose at desired levels. The number of medical devices 26₁-26_{N} may alternatively or additionally include one or more sensors or sensing systems that are external to the user's body and/or sensor techniques for providing information relating to the physiological condition of the user. Examples of such sensors or sensing systems may include, but should not be limited to, a glucose strip sensor/meter, a body temperature sensor, a blood pressure sensor, a heart rate sensor, or the like. In implementations that include an external glucose sensor, the system 10 may be a semi closed-loop system operable in a conventional manner to deliver insulin, as appropriate, based on glucose information provided thereto by the user. Information provided by any such sensors and/or senor techniques may be communicated to the system 10 using any one or more conventional wired or wireless communication technique.

In a second example implementation of the system 10, the electronic device 12 is provided in the form of a handheld remote device, such as a PDA or other handheld device. In this example, the number of medical devices 26₁-26_{N} include at least one conventional implantable or externally worn drug pump. In one embodiment of this example, an insulin pump is configured to controllably deliver insulin to the user's body. In this embodiment, the insulin pump is configured to wirelessly transmit information relating to insulin delivery to the handheld device 12. The handheld device 12 is configured to monitor insulin delivery by the pump, and may further be configured to determine and recommend insulin bolus amounts, carbohydrate intake, exercise, and the like. The system 10 may or may not be configured in this embodiment to provide for transmission of wireless information from the handheld device 12 to the insulin pump.

In an alternate embodiment of this example, the handheld device 12 is configured to control insulin delivery to the user by determining insulin delivery commands and transmitting such commands to the insulin pump. The insulin pump, in turn, is configured to receive the insulin delivery commands from the handheld device 12, and to deliver insulin to the user according to the commands. The insulin pump, in this embodiment, may or may not further process the insulin pump commands provided by the handheld unit 12. In any case, the system 10 will typically be configured in this embodiment to provide for transmission of wireless information from the insulin pump back to the handheld device 12 to thereby allow for monitoring of pump operation. In either embodiment of this example, the system 10 may further include one or more implanted and/or external sensors of the type described in the previous example.

Those skilled in the art will recognize other possible implementations of a closed-loop or semi-closed loop diabetes control arrangement using at least some of the components of the system 10 illustrated in FIG. 1. For example, the electronic device 12 in one or more of the above examples may be provided in the form of a laptop, notebook or personal computer configured to communicate with one or more of the medical devices 26₁-26_{N}, at least one of which is an insulin pump, to monitor and/or control the delivery of insulin to the user. As another example, the system 10 may further include a remote device (not shown) configured to communicate with the electronic device 12 and/or one or more of the medical devices 26₁-26_{N}, to control and/or monitor insulin delivery to the patient. The remote device may reside in a caregiver's office or other remote location, and communication between the remote device and any component of the system 10 may be accomplished via an intranet, internet (e.g., world-wide-web), cellular, telephone modem, RF, or other communication link. Any one or more conventional internet protocols may be used in such communications. Alternatively or additionally, any conventional mobile content delivery system; e.g., short message system (SMS), or other conventional message schema may be used to provide for communication between devices comprising the system 10. In any case, any such other implementations are contemplated by this disclosure.

Generally, the concentration of glucose in a person with diabetes changes as a result of one or more external influences such as meals and/or exercise, and may also change resulting from various physiological mechanisms such as stress, menstrual cycle and/or illness. In any of the above examples, the system 10 responds to the measured glucose by determining the appropriate amount of insulin to administer in order to maintain normal blood glucose levels without causing hypoglycemia. In some embodiments, the system 10 is implemented as a discrete system with an appropriate sampling rate, which may be periodic, aperiodic or triggered, although other continuous (analog) systems or hybrid systems may alternatively be implemented as described above.

As one example of a conventional diabetes control system, one or more software algorithms may include a collection of rule sets which use (1) glucose information, (2) insulin delivery information, and/or (3) subject inputs such as meal intake, exercise, stress, illness and/or other physiological properties to provide therapy, etc., to manage the user's glucose level. The rule sets are generally based on observations and clinical practices as well as mathematical models derived through or based on analysis of physiological mechanisms obtained from clinical studies. In the example system, models of insulin pharmacokinetics and pharmacodynamics, glucose pharmacodynamics, meal absorption and exercise responses of individual patients are used to determine the timing and the amount of insulin to be delivered. A learning module may be provided to allow adjustment of the model parameters when the patient's overall performance metric degrades (e.g., adaptive algorithms, using Bayesian estimates, may be implemented). An analysis model may also be incorporated which oversees the learning to accept or reject learning. Adjustments are achieved utilizing heuristics, rules, formulae, minimization of cost function(s) or tables (e.g., gain scheduling).

However, the human metabolism is complex and not fully understood. The solution space of managing glucose in daily life is currently limited. Day to day variability, incorrect or inaccurate input, device failures, physiological changes, exercise, stress, illness, etc. are known to produce changes in a diabetic person's condition. The working assumptions with conventional diabetes control systems are that the various device components are working correctly, and that the methodology or logic or process of determining therapy conforms to assumptions of operation. These assumptions are generally not accurate with actual diabetes control systems, and physical implementations of conventional diabetes control systems will generally encounter failure modes that the system cannot correct. Such failure modes may be detectable by the diabetes control system, while others may be detectable only by the user.

The following is a list of example failure modes that may be detectable by the diabetes control system. This list is not intended to be exhaustive or limiting, but is instead provided only by way of example.

1. Measurement Drift Error

Measurement drift is typically corrected in diabetes control systems with recalibration from time to time. The relation between the measured glucose (G_{M}) and true glucose (G) can be modeled according to the equation G_{M}=G+e, where e is the measurement error. If left unchecked, the error, e, may lead to unacceptable inaccuracies in G_{M}. There maybe one or more reasons for the inability of the system to correct glucose measurements.

2. Algorithm Models and Their Parameters

Models within the system typically use an approximation of the subject and device components to determine the therapy. The structures and parameters of the models define the anticipated behavior. However, the assumptions of the models may be inaccurate; the internal states of the models may not match with the actual subject, thereby leading to performance errors.

One example model, and potential sources of performance errors associated therewith, is a meal model. Errors in predicting meal absorption characteristics may result from inaccuracies in the dynamic behavior described by the shape of the user's carbohydrate absorption profile. Errors in timing as well as in shape of the profile may cause the diabetes control system to drive the user's glucose level toward hyperglycemic or hypoglycemic conditions. Similar considerations and error sources exist with respect to glucose measurement subcutaneous models, insulin absorption subcutaneous models (for various insulin types), exercise models, stress models glucose-insulin dynamics, and the like.

3. Feedback Systems

Any of a variety of conventional controller design methodologies, such as PID systems, full state feedback systems with state estimators, output feedback systems, LQG controllers, LQR controllers, eigenvalue/eigenstructure controller systems, and the like, could be used to design algorithms to perform physiological control. They typically function by using information derived from physiological measurements and/or user inputs to determine the appropriate control action to use. While the simpler forms of such controllers use fixed parameters (and therefore rules) for computing the magnitude of control action, the parameters in more sophisticated forms of such controllers may use one or more dynamic parameters. The one or more dynamic parameters could, for example, take the form of one or more continuously or discretely adjustable gain values. Specific rules for adjusting such gains could, for example, be defined either on an individual basis or on the basis of a patient population, and in either case will typically be derived according to one or more mathematical models. Such gains are typically scheduled according to one or more rule sets designed to cover the expected operating ranges in which operation is typically nonlinear and variable, thereby reducing sources of error. Errors in such feedback systems are, however, present, and therefore may accumulate and lead to unacceptable system inaccuracies.

4. Model Based Control System

Models describing the patient, for example, can be constructed as a black box wherein equations and parameters have no strict analogs in physiology. Rather, such models may instead be representations that are adequate for the purpose of physiological control. The parameters are typically determined from measurements of physiological parameters such as blood glucose, insulin concentration, and the like, and from physiological inputs such as food intake, alcohol intake, insulin doses, and the like, and also from physiological states such as stress level, exercise intensity and duration, menstrual cycle phase, and the like. These models are used to estimate current glucose or to predict future glucose values. Insulin therapy is derived by the system based on the model's ability to predict glucose for various inputs. Other conventional modeling techniques may be additionally or alternatively used including for example, but not limited to, building models from first principles. Errors in any such model types may result from a variety of causes such as incorrect estimation of model parameters, parameters that are non-linear and/or time varying, unmodeled system dynamics, incorrect dynamics, and the like.

5. Miscellaneous Factors Affecting Controller Performance

Errors arise from delays in action response, delays in measuring glucose, processing delays, delays caused by system operation cycle step size, and the like.

It is also desirable to provide for the ability to recover from situations that the system 10 does not or cannot detect as failures. For example, as a result of one or more of the above-described system error sources, the system 10 may drive the user's insulin sufficiently toward hyperglycemia or hypoglycemia that the user identifies or realizes the resulting symptoms even though the system 10 does not indicate any errors or failure modes. System errors/failures and/or user symptoms may be accelerated or decelerated as a result of the user's physiological state including, for example, illness, stress and the like.

The system 10 provides for user intervention in the diabetes control arrangement of the type or types described hereinabove. In particular, the input device 18 includes one or more user intervention input mechanisms that allow the user to intervene in the controlled insulin delivery algorithm being executed by a diabetes control arrangement in a manner that allows the insulin delivery algorithm to continue executing without resetting or otherwise disabling the algorithm and/or system. By appropriate selection/activation of the one or more user intervention input mechanisms, the user can take corrective action and then either allow the insulin delivery algorithm to act upon the corrective action (optionally with or without a delay) by including the corrective action in the execution of the insulin delivery algorithm, or to disregard, and not act upon, the corrective action by excluding the corrective action in the execution of the insulin delivery algorithm. In either case, though, the user enters the corrective action into the system 10. In one embodiment, the input device 18 includes two user-selectable buttons. By pressing one of the two user-selectable buttons, the user can intervene in the diabetes control arrangement, take corrective action and then allow the insulin delivery algorithm being executed to act upon the corrective action. By pressing the other of the two user-selectable buttons, the user can intervene in the diabetes control arrangement and take corrective action with the corrective action being excluded from the insulin delivery algorithm being executed. In either case, the corrective action is entered into the database in the memory unit or other data storage device 16. Also, in either case the insulin delivery algorithm continues to execute, and may also process the user intervention information depending upon appropriate selection of the user intervention input mechanism.

In an alternate embodiment, the display 20 includes a graphical user interface (GUI) that allows the user to select, at will, either of two user-selectable display icons. Selecting either of the two display icons will, in this embodiment, have the same effect as the selecting either of the two user-selectable buttons in the previous example. It will be understood that more, fewer, and/or other user-selectable input mechanisms may be provided to allow the user to intervene, at will, in the diabetes control arrangement, and to select between allowing the system 10 to act upon the corrective action taken in the intervention and having the system 10 disregard the corrective action taken in the intervention. Any such alterative user-selectable mechanisms are contemplated by this disclosure.

The user may intervene in the diabetes control arrangement, as just described, for the purpose of taking either of two possible corrective actions; namely, taking action to reduce the user's glucose level or taking action to increase the user's glucose level. Conventional mechanisms for reducing the user's glucose level include, but are not limited to, dispensing insulin into the user's body, such as in the form of a bolus and exercising. Conventional mechanisms for increasing the user's glucose level include, but are not limited to, ingesting carbohydrates and dispensing glucogen into the user's system. Either corrective action taken by the user is independent of the system logic and consideration of devices within the system 10. Such user intervention allows the system 10 to continue operation under the insulin delivery algorithm while also allowing the system 10 to recover without necessarily requiring a system reset.

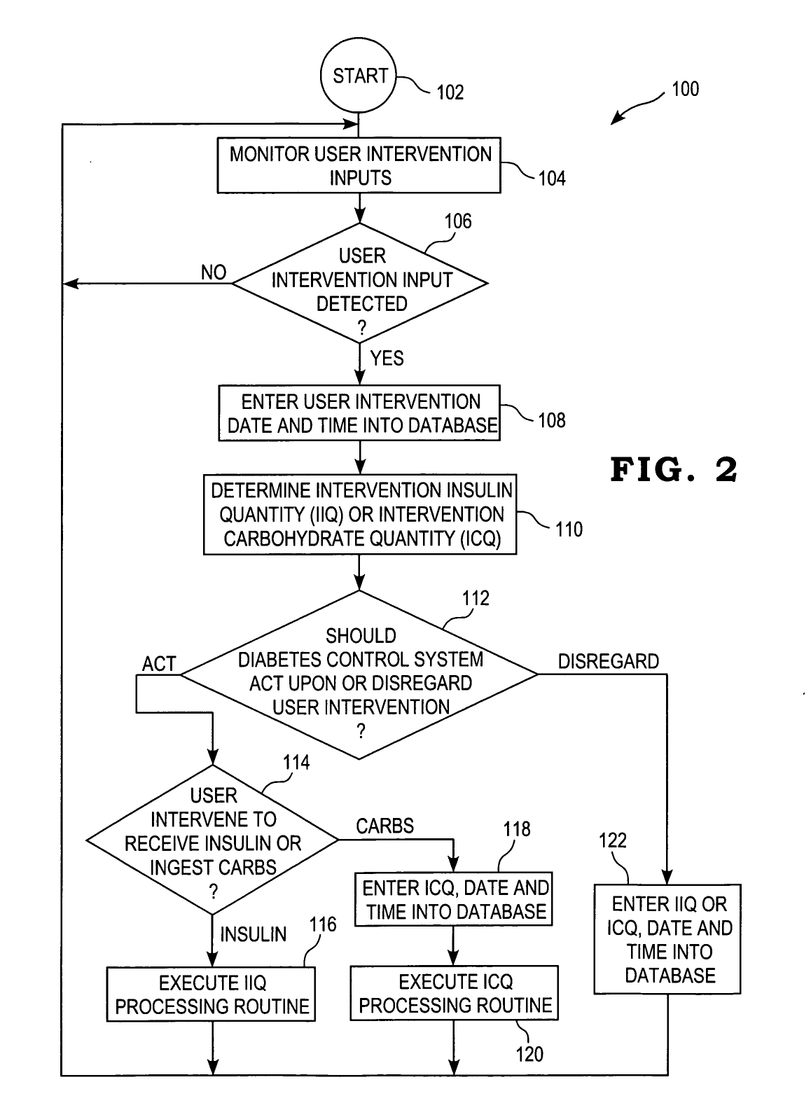

Referring now to FIG. 2, a flowchart of one illustrative embodiment of a software algorithm 100 for providing for user intervention in a diabetes control arrangement is shown. The algorithm 100 will typically be stored in the memory unit or other data storage device 16, and will be executed by the processor 14. In the illustrated embodiment, it will be understood that the processor 14 will be, simultaneously or in tandem, executing one or more conventional insulin delivery algorithms configured to manage or control delivery of insulin to the user, and that the algorithm 100 will therefore be executed by the processor 14 as an independent algorithm. Alternatively, the algorithm 100 and the one or more conventional insulin delivery algorithms may be executed by different processors in an embodiment of the system 10 that includes multiple processors. In any case the algorithm 100 will be described for purposes of this document as being executed by the processor 14. In this description, it will be understood that the algorithm 100 treats user interventions as asynchronous occurrences requiring immediate attention, as compared with synchronous, e.g., periodic, events that the system 10 normally manages in accordance with the one or more insulin delivery algorithms. The algorithm 100 begins at step 102, and thereafter at step 104 the processor 14 is operable to monitor the one or more user intervention input mechanisms described hereinabove. Thereafter at step 106, the processor 14 is operable to determine whether one of the one or more user intervention input mechanisms has been selected or activated. If not, algorithm execution loops back to step 104. If so, this means that the user has manually selected one of the two user intervention input mechanisms, and algorithm execution advances to step 108 where the processor 14 is operable to enter the user intervention event, date and time into the database contained within the memory unit or other data storage device 16. Thereafter at step 110, the processor 14 is operable to determine either an intervention insulin quantity (IIQ) or an intervention carbohydrate quantity (ICQ).

As described hereinabove, the user may intervene in the diabetes control arrangement, as just described, for the purpose of taking either of two possible corrective actions; either by taking action to decrease the user's glucose level, e.g., by receiving insulin, such as in the form of a bolus, and/or via one or more other conventional glucose decreasing mechanisms, or by taking action to increase the user's glucose level, e.g., by ingesting carbohydrates and/or via one or more other conventional glucose increasing mechanisms. In cases where the user chooses to intervene by taking additional insulin, the user may do so via any conventional technique. Examples include, but are not limited to, manually overriding the system 10 in a conventional manner to direct the system 10 to deliver a specified amount of insulin, programming the system 10 in a conventional manner to deliver the specified amount of insulin, manually injecting the specified amount of insulin via a syringe, or the like. In any case, the user enters the specified amount of insulin into the system 10 via an appropriate one of the input devices 18, and the processor 14 executes step 110 by receiving the specified amount of insulin, or intervention insulin quantity (IIQ), from the input device 18. In cases where the user chooses to intervene by ingesting carbohydrates, the user enters the quantity of carbohydrates that were ingested into the system 10 via an appropriate input device 18. The processor 14 executes step 110 in this case by receiving the intervention carbohydrate quantity (ICQ) from the input device 18. In either case, it will be understood that the algorithm 100 will also typically include one or more steps providing a timeout mechanism that allows the algorithm 100 to continue execution after a predefined time period when the user fails to enter, or incompletely enters, IIQ or ICQ information at step 110. Any such one or more steps would be a mechanical exercise for a skilled algorithm designer.

From step 110, the algorithm 100 advances to step 112 where the processor 14 is operable to determine whether the system, 10 should act upon or disregard the user intervention in the form of corrective action taken at step 110. In the illustrated embodiment, the processor 14 is operable to execute step 112 in accordance with the particular user intervention input detected at step 106. More specifically, if the user intervened in the operation of the system 10 by selecting a user intervention input designated for action, then the algorithm 100 advances to step 114 where the system 10 is operable to act upon or process the corrective action taken by the user. At step 114, the processor 14 is operable to determine whether the corrective action detected at step 106 corresponds to administering of insulin or ingestion of carbohydrates. The processor 14 is operable to execute step 114, in the illustrated embodiment, by determining the nature of the parameter received at step 110. Specifically, if the parameter IIQ is received at step 110, then algorithm execution advances from step 114 to step 116 where the processor 14 executes an IIQ processing routine, which allows the one or more insulin delivery algorithms being executed by the processor 14 to include the intervention insulin quantity, IIQ, in the execution thereof under the direction of the IIQ processing routine. If, on the other hand, the parameter ICQ is received at step 110, then algorithm execution advances from step 114 to step 118 where the processor 14 is operable to time and date stamp ICQ and then enter this data into the database portion of the memory unit or other data storage device 16. Following step 118, the processor 14 is operable at step 120 to execute an ICQ processing routine, which allows the one or more insulin delivery algorithms being executed by the processor 14 to include the intervention carbohydrate quantity, ICQ, in the execution thereof under the direction of the ICQ processing routine. If, at step 112, the user intervened in the operation of the diabetes control system 10 by selecting a user intervention input designated for inaction, then the algorithm advances from step 112 to step 122 where the processor 14 is operable to time and date stamp the corrective action, IIQ or ICQ, and then enter this data into the database portion of the memory unit or other data storage device 16. The processor 14, in this case, excludes the corrective action, IIQ or ICQ, from the one or more insulin delivery algorithms being executed by the processor 14, so that the system 10 does not act upon the corrective action taken by the user. The algorithm 100 loops from any of steps 116, 120 and 122 back to step 104.

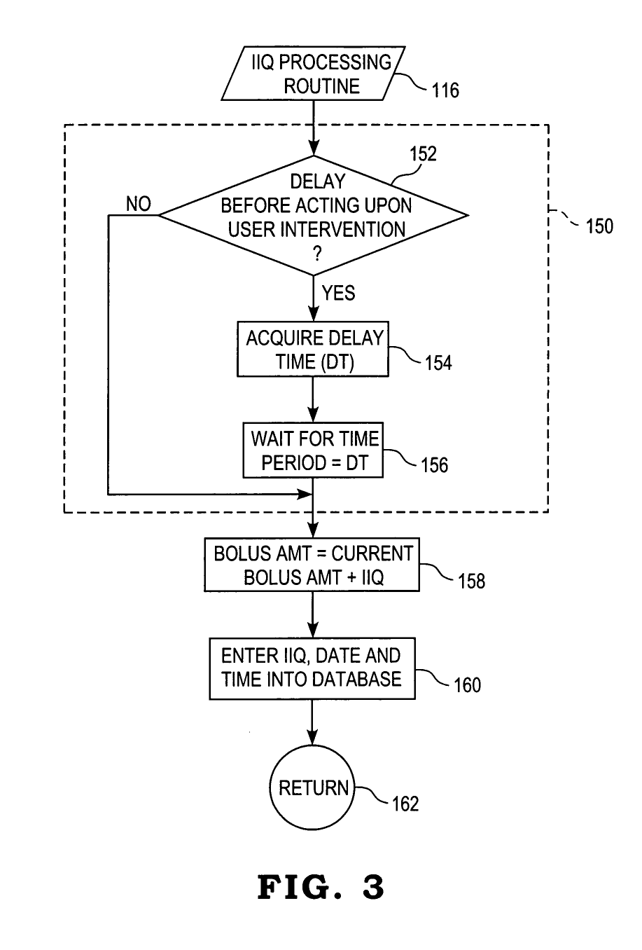

Referring now to FIG. 3, a flowchart of one illustrative embodiment of the IIQ processing routine of step 116 of the algorithm 100 of FIG. 2 is shown. In the illustrated embodiment, the routine 116 may include an optional step 150 that allows for a selectable delay period prior to acting upon IIQ. For example, step 150 may comprise step 152 where the processor 14 is operable to determine whether to delay before acting upon IIQ. In one embodiment, the processor 14 is operable to execute step 152 by prompting the user for a delay time, DT. If the user enters zero, via a suitable input device 18, then execution of the routine advances to step 158. If, on the other hand, if the user enters a positive value, then execution of the routine 116 advances to step 154 where the processor 14 is operable to receive the delay time, DT, entered by the user. In an alternate embodiment, the processor 14 may be operable to execute step 152 by prompting the user answer yes or no to whether to delay before processing IIQ. If the user enters no, via a suitable input device 18, execution of the routine 116 advances to step 158. If, on the other hand, the user answers yes at step 152, the processor 14 then prompts the user at step 154 to enter, via a suitable input device 18, a delay time value, DT. In any case, execution of the routine 116 advances from step 154 to step 156 where the processor 14 is operable to wait for a time period equal to DT before advancing to step 158. The optional step 150 may further include one or more steps designed to allow the user to cancel the intervention, and/or to accept/acknowledge one or more additional user interventions, during the delay period, DT. Any such one or more steps would be a mechanical exercise for a skilled algorithm designer. It will be understood that, in embodiments where the user specifies the delay time, DT, the routine 116 will also typically include one or more steps providing a timeout mechanism that allows the routine 116 to continue execution after a predefined time period when the user fails to enter, or incompletely enters, the delay time, DT, at step 154. Any such one or more steps would be a mechanical exercise for a skilled algorithm designer.

At step 158, the processor 14 is operable in the illustrated embodiment of the IIQ processing routine 116 to process the intervention insulin quantity, IIQ, by adding IIQ to any currently scheduled bolus amount, where “currently” is defined for purposes of step 158 as the point in the execution of the insulin delivery algorithm at which step 158 of the routine 116 is also executed. If some positive amount of insulin bolus is currently scheduled for delivery to the user, the processor 14 is operable at step 158 to add IIQ to the positive amount of insulin bolus already scheduled for delivery to the user. If, on the other hand, no bolus amount is currently scheduled, i.e., the current bolus amount is zero, the processor 14 is operable to schedule a bolus amount of IIQ according to the insulin delivery algorithm being executed by the processor 14. The system 10 is thereafter operable to manage delivery of the insulin bolus to the user according to the one or more insulin delivery algorithms being executed by the processor 14. In alternate embodiments of the IIQ processing routine 116, the processor 14 may be configured to control delivery of an insulin bolus in the amount of IIQ before, during or after delivery of any currently scheduled insulin bolus. In any case, following execution of step 158 the processor 14 is operable at step 160 to date and time stamp IIQ, and to then enter the date and time stamped IIQ value into the database portion of the memory unit or other data storage device 16. The routine 116 returns thereafter at step 162 to the algorithm 100 of FIG. 2. It will be understood that in one or more embodiments of the system 10, it may be desirable to synchronize date and/or time stamping of IIQ with a reference date and/or time using one or more conventional date and/or time synchronization techniques. It will also be understood that the IIQ data is date and time stamped, and then stored in the memory unit or other data storage device 16, at or near the time that the intervention insulin quantity, IIQ, is scheduled for delivery, or actually delivered, to the user. In the embodiment of the routine 116 illustrated in FIG. 3, this step occurs after the optional delay step 150. In other embodiments, the appropriate time to date and time stamp IIQ and enter this information into the memory unit or other data storage device 16 will become apparent. As one specific example, in embodiments where the intervention insulin quantity, IIQ, is manually administered, it will be appropriate to date and time stamp the IIQ data at or near the time that the intervention insulin quantity is actually administered; e.g., such as directly following step 110 of the algorithm 100. Similar considerations apply to the date, time stamping and storage of the intervention carbohydrate quantity, ICQ.

The routine 116 of FIG. 3 will typically be called and executed when the user intervenes, via the algorithm 100 of FIG. 2, in the operation of the diabetes control arrangement as a result of a high glucose event or condition. A high glucose event or condition is defined, in one embodiment, by a high glucose threshold value, a minimum duration above the threshold value, and the rate of change of glucose defined by a maximum threshold rate and a minimum threshold rate. The threshold values may be based on predicted values or measured values or a combination of both. In any case, the user may execute a high glucose intervention typically as a result of any one or more of the following occurrences:

1. The system 10 has flagged the user's glucose as exceeding a high glucose threshold value that was pre-set by a default setting,

2. The system 10 has flagged the user's glucose as exceeding a high glucose threshold value set by a health care professional,

3. The system 10 has flagged the user's glucose as exceeding a high glucose threshold value set by the user, user's parent or guardian, or other care giver,

4. The user, or third party, has identified the high glucose event based on an independent physical measurement of the user's glucose level,

5. The user, or third party, has identified the high glucose event based on independent physiological symptoms/indicators, or

6. The system 10 has identified the high glucose event based on analysis according to one or more predictive models.

The user may react to the high glucose event by administering an intervention insulin amount, such as in the form of a bolus, as described above. If the user chooses not to allow the processor 14 to act upon this administered insulin quantity, IIQ, the insulin delivery algorithm being executed by the diabetes control system 10 will not reduce this amount of insulin from future control actions. If, however, the user chooses to allow the processor 14 to act upon the administered insulin quantity, IIQ, the processor 14 schedules delivery of an insulin bolus in the amount of IIQ.

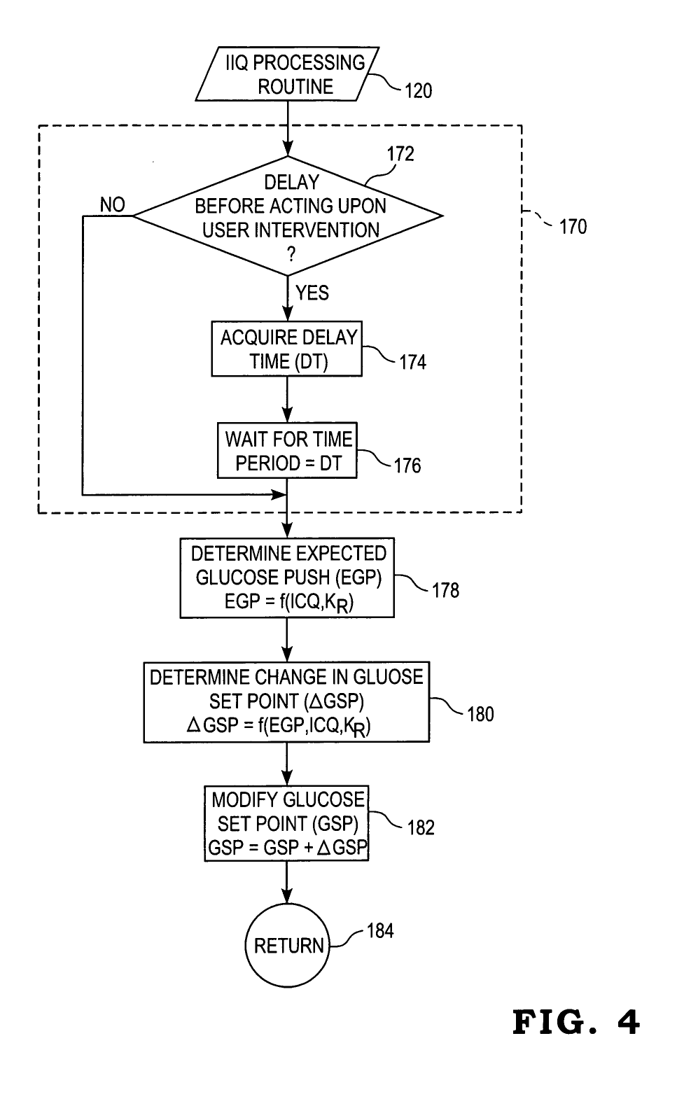

Referring now to FIG. 4, a flowchart of one illustrative embodiment of the ICQ processing routine of step 120 of the algorithm 100 of FIG. 2 is shown. In the illustrated embodiment, the routine 120 may include an optional step 170 that allows for a selectable delay period prior to acting upon ICQ. For example, step 170 may comprise step 172 where the processor 14 is operable to determine whether to delay before acting upon ICQ. In one embodiment, the processor 14 is operable to execute step 172 by prompting the user for a delay time, DT. If the user enters zero, via a suitable input device 18, then execution of the routine advances to step 178. If, on the other hand, if the user enters a positive value, then execution of the routine 120 advances to step 174 where the processor 14 is operable to receive the delay time, DT, entered by the user. In an alternate embodiment, the processor 14 may be operable to execute step 172 by prompting the user answer yes or no to whether to delay before processing ICQ. If the user enters no, via a suitable input device 18, execution of the routine 120 advances to step 178. If, on the other hand, the user answers yes at step 172, the processor 14 then prompts the user at step 174 to enter, via a suitable input device 18, a delay time value, DT. In any case, execution of the routine 120 advances from step 174 to step 176 where the processor 14 is operable to wait for a time period equal to DT before advancing to step 178. The optional step 170 may further include one or more steps designed to allow the user to cancel the intervention, and/or to accept/acknowledge one or more additional user interventions, during the delay period, DT. Any such one or more steps would be a mechanical exercise for a skilled algorithm designer. It will be understood that, in embodiments where the user specifies the delay time, DT, the routine 120 will also typically include one or more steps providing a timeout mechanism that allows the routine 120 to continue execution after a predefined time period when the user fails to enter, or incompletely enters, the delay time, DT, at step 174. Any such one or more steps would be a mechanical exercise for a skilled algorithm designer.

At steps 178-182, the processor 14 is operable to process the intervention carbohydrate quantity, ICQ, according to the one or more insulin delivery algorithms being executed by the processor 14. In the illustrated embodiment, the processor 14 is operable to process the intervention carbohydrate quantity, ICQ, by first determining at step 178 an expected glucose push function, EGP, which is a normalized representation of an expected profile of glucose push and the normalized function is, in this example, scaled by ICQ and K_{R}, where K_{R} corresponds to glucose rise per gram of carbohydrates. The expected glucose push function, EGP, is a normalized time-based glucose push function resulting from the intake of fast-acting carbohydrates, ICQ. Following step 178, the processor 14 is operable at step 180 to determine a change in the current glucose target value, or glucose set point, ΔGSP, as a function of EGP, ICQ and K_{R}. More specifically, the change in the glucose set point, ΔGSP, is determined as a product of a linearly decreasing gain term, [1−(Δt/T_{D})], ICQ, K_{R} and the cumulative sum of EGP over time, where At is the elapsed time from the instant of intervention and T_{D} is the duration over which the intervention action will last. In particular, ΔGSP=[1−(Δt/T_{D})]*ICQ*K_{R}*EGP(Δt). Following step 180, the processor 14 is operable at step 182 to determine the glucose target value or set point, GSP, as a sum of the current glucose set point and the change in the glucose set point, or GSP=GSP+ΔGSP. The routine 120 returns thereafter at step 186 to the algorithm 100 of FIG. 2. It will be understood that in one or more embodiments of the system 10, it may be desirable to synchronize date and/or time stamping of ICQ with a reference date and/or time using one or more conventional date and/or time synchronization techniques.

In the embodiment illustrated herein, the intervention insulin carbohydrate quantity, ICQ, is typically expected to be provided in the form of fast-acting carbohydrates, as this term is commonly understood in the art. In this embodiment, ICQ will generally be provided in the form of one or more fast-acting carbohydrate foods and/or liquids, or may alternatively be provided in pill or chewable tablet form, or may alternatively still be provided in the form of an injectable drug, such as glucogen. In alternate embodiments of the system 10, the algorithm 100 and/or routine 120 may be modified to allow the user to intervene by ingesting or otherwise receiving fast-acting carbohydrates or by ingesting or otherwise receiving slower-acting carbohydrates. In such embodiments, the system 10, algorithm 100 and routine 120 may be modified to distinguish between carbohydrates ingested or otherwise received in the form of fast-acting carbohydrates and slower-acting carbohydrates. In such embodiments, the system 10 will provide for user input of such information, the algorithm 100 may allow the user to input the type of carbohydrates being ingested or otherwise received, and the routine 120 may respond to the type of carbohydrates ingested by the user by, for example, selecting, calculating or otherwise determining an appropriate ΔGSP function based upon carbohydrate type. Any such modifications to the system 10, algorithm 100 and/or routine 120 would be a mechanical step for a skilled artisan.

The routine 120 of FIG. 4 will typically be called and executed when the user intervenes, via the algorithm 100 of FIG. 2, in the operation of the system 10 as a result of a low glucose event or condition. A low glucose event or condition is defined, in one embodiment, by a lower glucose threshold value and the rate of change of glucose defined by a maximum threshold rate and a minimum threshold rate. The threshold values may be based on predicted values or measured values or a combination of both. In any case, the user may execute a low glucose intervention typically as a result of any one or more of the following occurrences:

1. The system 10 has flagged the user's glucose as exceeding a low glucose threshold value that was pre-set by a default setting,

2. The system 10 has flagged the user's glucose as exceeding a low glucose threshold value set by a health care professional,

3. The system 10 has flagged the user's glucose as exceeding a low glucose threshold value set by the user, user's parent or guardian, or other care giver,

4. The user, or other third party, has identified the low glucose event based on an independent physical measurement of the user's glucose level,

5. The user, or other third party, has identified the low glucose event based on independent physiological symptoms/indicators, or

6. The system 10 has identified the low glucose event based on analysis according to one or more predictive models.

The user may react to the low glucose event by ingesting or otherwise receiving a carbohydrate composition, such as in the form of fast-acting carbohydrates foods and/or liquids, one or more glucose increasing pills or chewable tablets and/or a glucose increasing drug. This action is intended to increase the user's glucose level back to a normal glycemic range. If the user chooses not to allow the processor 14 to act upon the intervention carbohydrate quantity, ICQ, by excluding ICQ from the insulin delivery algorithm being executed by the processor 14, the system 10 will not attempt to counteract the resulting increase in glucose by recommending additional insulin. If, however, the user chooses to allow the processor 14 to act upon the intervention carbohydrate quantity, ICQ, by including ICQ in the execution of the insulin delivery algorithm being executed by the processor 14, the system 10 may attempt to counteract this glucose push by recommending delivery of additional insulin. Steps 178-182 of the routine 120 of FIG. 4 thus add a time-decaying function to the existing glucose target or set point, GSP. By modifying the glucose set point GSP initially by an amount equal to the expected rise EGP, the system 10 will not attempt to counteract the glucose rise attributed to the intake of fast-acting carbohydrates. The time-decaying function ΔGSP allows the modified glucose set point, GSP, to return to its original set point after the passage of an amount of time. It will be understood that other conventional techniques may be used to allow the one or more insulin delivery control algorithms being executed by the processor 14 to gradually return to normal operation following user intervention in the form of ingesting or otherwise receiving a glucose-increasing composition. As an example of one such alternate technique, the system 10 may be configured to temporarily modify the rate of allowable insulin rise, and to allow the rate of allowable insulin rise to return to normal after the passage of some amount of time. This and any other such alternate technique for allowing the one or more insulin delivery control algorithms being executed by the processor 14 to gradually return to normal operation following user intervention in the form of ingesting or otherwise receiving a glucose-increasing composition is contemplated by this disclosure.

An example of one situation where it may be appropriate for the user to instruct the system 10 to disregard a user's intervention occurs with a meal-related glucose rise resulting from ingesting meals of unknown or partially known composition. If the dynamic response of the system 10 is not matched properly with the meal composition, the system 10 may inadvertently push the diabetic subject into hypoglycemic condition. User intervention, as described herein, allows the handling of unknown dynamics; e.g., unknown meal load, in a controlled manner.

A meal is typically covered with the system 10, under the control of the insulin delivery algorithm being executed by the processor 14, by controllably dispensing insulin doses based on predicted meal absorption profiles. This insulin distribution is determined so as to best minimize the glucose rise, and to bring the glucose to the target glucose level as quickly as possible with minimal undershoot. However clinical data have shown large absorption variability due to complexity associated with meal composition, persistence of prior meal affects and influences, inaccuracy in measurement techniques of meal size, style of meal consumption, etc. Such large variability, if observed, may be best handled, for example, with the user intervention system described herein by riding out the transient uncertainty. Other conventional techniques for responding to such variability using one or more conventional techniques.

The glucose rise to meal intake cannot be removed completely. This is expected since delays in peak insulin action may typically be about 30-60 minutes. The insulin dosage obtained is optimized to minimize glucose rise due to the meal. A meal-related target glucose zone is defined around the meal event as a region bounded by upper and lower target glucose boundaries. With respect to the defined target zone, the following four scenarios occur

1. Within Glucose Zone

If the predicted glucose value lies within the glucose zone boundaries, then the user's glucose is considered within acceptable limits. The processor 14 assumes, under the insulin delivery algorithm being executed by the processor 14, that the glycemic behavior is within acceptable limits and continues to recommend insulin with no correction for glucose deviation.

2. Above the Glucose Zone

If the predicted glucose lies above the upper glucose boundary, then the user is considered as under-delivered in insulin. The processor 14 computes, under control of the insulin delivery algorithm being executed by the processor 14, the deviation in glucose with respect to the upper glucose boundary. The basal controller action accounts for this deviation and will curb for this unaccounted rise.

3. Below the Glucose Zone

If the predicted glucose lies below the lower glucose boundary, then the user is considered as over-delivered in insulin. The processor 14 computes, under control of the insulin delivery algorithm being executed by the processor 14, the deviation in glucose with respect to the lower glucose boundary. The basal controller action accounts for this deviation and will curb for this unaccounted fall.

4. No Glucose Update

The target zone covers the rise and fall of anticipated meal related response. A special case arises when glucose information in the system 10 is not updated; e.g., when a new measurement has not been received since the previous measurement or is not receive within a pre-scheduled interval. With no update on glucose measurement the predicted glucose for the current control cycle is a glucose value without accounting for the meal related rise or fall in glucose. The target zone boundaries however are function of time. This generally means that the predicted glucose is lower when meal effects on the body are begging to occur, and is higher when meal effects on the body are tapering off. This effect is accentuated with rising and falling meal zone boundaries. The insulin delivery algorithm being executed by the processor 14 handles this case by holding the boundary limits last used with the last received glucose measurement. These upper and lower target values are held fixed for all future control cycles, until a new measurement is available.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, the concepts described herein may be applicable to other medical control arrangements having a processor executing a drug delivery algorithm forming part of the medical control arrangement. In any such system, the processor may be responsive to the first user intervention signal to include an intervention therapy value in the execution of the drug delivery algorithm, and responsive to the second user intervention signal to exclude the intervention therapy value from the execution of the drug delivery algorithm. The intervention therapy value may correspond to various medical treatments administered to and/or carried out by the user including for example, but not limited to, delivery of one or more drugs, such as insulin, glucogen or other drugs, administering one or more other drugs and/or carrying one or more acts that have an affect opposite that of delivering the one or more drugs, ingesting carbohydrates, executing one or more physical exercises, or the like. Other examples will occur to those skilled in the art, and any such other examples are contemplated by the present disclosure.

As another example, the electronic device 12 of FIG. 1 may include several selectable input mechanisms for acting upon and not acting upon user interventions. As one specific example, the device 12 may include multiple “preset” input mechanisms that allow the user to select a preset amount of insulin from a number of selectable preset insulin amounts, for delivery to the user.

As yet another example, the system 10 may receive multiple user intervention requests, such as when delaying action pursuant to optional steps 150 or 170 of the routines 116 and 120 respectively. In such cases, the multiple requests may be executed as a group. Alternatively, the system 10 may include one or more priority algorithms configured to prioritize the various user intervention events according to one or more predetermined, programmable or user-selectable criteria.

What is claimed is:

1. A system providing for user intervention in a diabetes control arrangement, the system comprising:
means responsive to user selection thereof for producing one of a first and a second user intervention signal, and
a processor executing an insulin delivery algorithm forming part of the diabetes control arrangement, the processor responsive to the first user intervention signal to include one of an intervention insulin quantity and an intervention carbohydrate quantity in the execution of the insulin delivery algorithm, and responsive to the second user intervention signal to exclude the one of the intervention insulin quantity and the intervention carbohydrate quantity from the execution of the insulin delivery algorithm.

2. The system of claim 1 wherein the processor is configured to continue uninterrupted execution of the insulin delivery algorithm regardless of whether the first or second user intervention signal is produced.

3. The system of claim 1 further including means for providing the one of the intervention insulin quantity and the intervention carbohydrate quantity to the processor.

4. The system of claim 1 wherein the processor is responsive to the first user intervention signal to process the intervention insulin quantity by adding the intervention insulin quantity to a current insulin bolus amount.

5. The system of claim 4 wherein the processor is further responsive to the first user intervention signal to command administration of the combination of the intervention insulin quantity and the current insulin bolus amount to the user.

6. The system of claim 4 wherein the current insulin bolus amount is a positive-valued insulin bolus amount.

7. The system of claim 4 wherein the current insulin bolus amount is a zero-valued insulin bolus amount.

8. The system of claim 1 wherein the processor is responsive to the first user intervention signal to process the intervention carbohydrate quantity by modifying a blood glucose target as a function of the intervention carbohydrate quantity.

9. The system of claim 1 further including a database having insulin delivery and intervention carbohydrate information stored therein, and wherein the processor is responsive to either of the first and second user intervention signals to enter the one the intervention insulin quantity and the intervention carbohydrate quantity into the database.

10. The system of claim 1 wherein the processor is operable to wait for a delay time prior to including the one of the intervention insulin quantity and the intervention carbohydrate quantity in the execution of the insulin delivery algorithm.

11. A method of allowing user intervention in a diabetes control arrangement, the method comprising:
executing an insulin delivery algorithm forming part of the diabetes control arrangement,
monitoring first and second user intervention mechanisms,
including one of an intervention insulin quantity and an intervention carbohydrate quantity in the execution of the insulin delivery algorithm in response to user selection of the first user intervention mechanism, and
excluding the one of the intervention insulin quantity and the intervention carbohydrate quantity from the execution of the insulin delivery algorithm in response to user selection of the second user intervention mechanism.

12. The method of claim 11 further including receiving the one of the intervention insulin quantity and the intervention carbohydrate quantity.

13. The method of claim 11 further including entering the one of the intervention insulin quantity and the intervention carbohydrate quantity into a database in response to user selection of either of the first and second user intervention mechanisms.

14. The method of claim 13 further including date and time stamping the one of the intervention insulin quantity and the intervention carbohydrate quantity prior to entry into the database.

15. The method of claim 11 further including waiting for a delay time after the user selection of the first user intervention mechanism and prior to including the one of the intervention insulin quantity and the intervention carbohydrate quantity in the execution of the insulin delivery algorithm.

16. A system providing for user intervention in a medical control arrangement, the system comprising:
a first user intervention mechanism responsive to user selection thereof to produce a first user intervention signal,
a second user intervention mechanism responsive to user selection thereof to produce a second user intervention signal, and
a processor executing a drug delivery algorithm forming part of the medical control arrangement, the processor responsive to the first user intervention signal to include an intervention drug quantity in the execution of the drug delivery algorithm, and responsive to the second user intervention signal to exclude the intervention drug quantity from the execution of the drug delivery algorithm.

17. The system of claim 16 further including means for receiving the intervention drug quantity.

18. The system of claim 16 wherein the medical control arrangement is a diabetes control arrangement, the drug delivery algorithm is an insulin delivery algorithm, and the intervention drug quantity is an intervention insulin quantity.

19. The system of claim 18 wherein the processor is responsive to the first user intervention signal to include the intervention insulin quantity in the execution of the insulin delivery algorithm by adding the intervention insulin quantity to a current insulin bolus amount.

20. The system of claim 19 wherein the processor is further responsive to the first user intervention signal to command administration of the combination of the intervention insulin quantity and the current insulin bolus amount to the user.

21. The system of claim 16 further including a database having drug delivery information stored therein,
wherein the processor is responsive to either of the first and second user intervention signals to enter the intervention drug quantity into the database.

22. The system of claim 21 wherein the processor is configured to date and time stamp the intervention drug quantity prior to entry into the database.

23. The system of claim 16 wherein the processor is operable to wait for a delay time prior to including the intervention drug quantity in the execution of the insulin delivery algorithm.

24. The system of claim 16 wherein the processor is configured to continue uninterrupted execution of the drug delivery algorithm regardless of whether the first or second user intervention signal is produced.

25. A method of allowing user intervention in a medical control arrangement, the method comprising:
executing a drug delivery algorithm forming part of the medical control arrangement,
monitoring first and second user intervention mechanisms,
including an intervention drug quantity in the execution of the drug delivery algorithm in response to user selection of the first user intervention mechanism, and
excluding the intervention drug quantity from the execution of the drug delivery algorithm in response to user selection of the second user intervention mechanism.

26. The method of claim 25 further including receiving the intervention drug quantity.

27. The method of claim 25 further including entering the intervention drug quantity into a database in response to user selection of either of the first and second user intervention mechanisms.

28. The method of claim 27 further including date and time stamping the intervention drug quantity prior to entry into the database.

29. The method of claim 25 further including waiting for a delay time after the user selection of the first user intervention mechanism and prior to including the intervention drug quantity in the execution of the drug delivery algorithm.

30. The method of claim 25 wherein the medical control arrangement is a diabetes control arrangement, the drug delivery algorithm is an insulin delivery algorithm and the intervention drug quantity is an insulin intervention quantity.

31. A system providing for user intervention in a medical control arrangement, the system comprising:
a first user intervention mechanism responsive to user selection thereof to produce a first user intervention signal,
a second user intervention mechanism responsive to user selection thereof to produce a second user intervention signal, and
a processor executing a drug delivery algorithm forming part of the medical control arrangement, the processor responsive to the first user intervention signal to include an intervention therapy value in the execution of the drug delivery algorithm, and responsive to the second user intervention signal to exclude the intervention therapy value from the execution of the drug delivery algorithm.

32. The system of claim 31 further including means for receiving the intervention therapy value.

33. The system of claim 31 wherein the medical control arrangement is a diabetes control arrangement, the drug delivery algorithm is an insulin delivery algorithm, and the intervention therapy value is an intervention insulin quantity.

34. The system of claim 31 wherein the medical control arrangement is a diabetes control arrangement, the drug delivery algorithm is an insulin delivery algorithm, and the intervention therapy value is an intervention carbohydrate quantity corresponding to a quantity carbohydrates recently intervention by the user.

35. The system of claim 34 wherein the processor is responsive to the first user intervention signal to include the intervention carbohydrate quantity in the execution of the insulin delivery algorithm by modifying a blood glucose target as a function of the intervention carbohydrate quantity.

36. The system of claim 31 further including a database having therapy value information stored therein,
wherein the processor is responsive to either of the first and second user intervention signals to enter the intervention therapy value into the database.

37. The system of claim 36 wherein the processor is configured to date and time stamp the intervention therapy value prior to entry into the database.

38. The system of claim 31 wherein the processor is operable to wait for a delay time prior to including the intervention therapy value in the execution of the drug delivery algorithm.

39. The system of claim 31 wherein the processor is configured to continue uninterrupted execution of the drug delivery algorithm regardless of whether the first or second user intervention signal is produced.

40. A method of allowing user intervention in a medical control arrangement, the method comprising:
executing a drug delivery algorithm forming part of the medical control arrangement,
monitoring first and second user intervention mechanisms,
including an intervention therapy value in the execution of the drug delivery algorithm in response to user selection of the first user intervention mechanism, and
excluding the intervention therapy value from the execution of the drug delivery algorithm in response to user selection of the second user intervention mechanism.

41. The method of claim 40 further including receiving the intervention therapy value.

42. The method of claim 40 further including entering the intervention therapy value into a database in response to user selection of either of the first and second user intervention mechanisms.

43. The method of claim 42 further including date and time stamping the intervention therapy value prior to entry into the database.

44. The method of claim 40 further including waiting for a delay time after the user selection of the first user intervention mechanism and prior to including the intervention therapy value in the execution of the drug delivery algorithm.

45. The method of claim 40 wherein the medical control arrangement is a diabetes control arrangement, the drug delivery algorithm is an insulin delivery algorithm and the intervention therapy value is an insulin intervention quantity.

46. The method of claim 40 wherein the medical control arrangement is a diabetes control arrangement, the drug delivery algorithm is an insulin delivery algorithm and the intervention therapy value is an intervention carbohydrate quantity corresponding to a quantity carbohydrates recently intervention by the user.
